(19) United States
(12) Patent Application Publication
(10) Pub. No.: US 2024/0180927 A1
(43) Pub. Date: Jun. 6, 2024

(54) Methods of Treating Testosterone Deficiency

(71) Applicant: Tolmar, Inc., Fort Collins, CO (US)

(72) Inventors: Robert E. Dudley, Murfreesboro, TN (US); Theodore Danoff, Philadelphia, PA (US); James A. Longstreth, Mundelein, IL (US)

(21) Appl. No.: 18/433,069

(22) Filed: Feb. 5, 2024

Related U.S. Application Data
(63) Continuation of application No. 18/451,557, filed on Aug. 17, 2023.
Application No. 18/451,557 is a continuation of application No. 17/235,439, filed on Apr. 20, 2021, now abandoned.
Application No. 17/235,439 is a continuation of application No. 16/720,194, filed on Dec. 19, 2019, now abandoned.
(60) Provisional application No. 62/782,865, filed on Dec. 20, 2018.

(51) Int. Cl. A61K 31/568; A61K 9/00; A61K 47/12; A61K 47/44; A61P 5/26

(52) U.S. Cl. CPC A61K 31/568; A61K 9/0053; A61K 47/12; A61K 47/44; A61P 5/26

(57) ABSTRACT

Methods of treating a testosterone deficiency or its symptoms with a pharmaceutical formulation of testosterone undecanoate are provided.

This application is a continuation of U.S. application Ser. No. 16/720,194, filed Dec. 19, 2019, which claims priority to U.S. provisional application No. 62/782,865, filed Dec. 20, 2018, the disclosures of which are incorporated by reference herein in their entiretics.

Provided are treatments for testosterone deficiency and methods utilizing oral formulations of testosterone undecanoate that optimize the serum testosterone concentration during chronic treatment.

Testosterone (T) is a primary androgenic hormone produced in the interstitial cells of the testes and is responsible for normal growth, development and maintenance of male sex organs and secondary sex characteristics (e.g., deepening voice, muscular development, facial hair, etc.). Throughout adult life, testosterone is necessary for proper functioning of the testes and its accessory structures, prostate and seminal vesicle; for sense of well-being; and for maintenance of libido, crectile potency.

Testosterone deficiency—insufficient secretion of T characterized by low total T concentrations—can give rise to medical conditions (e.g., hypogonadism) in males. Symptoms associated with male hypogonadism include impotence and decreased sexual desire, fatigue and loss of energy, mood depression, regression of secondary sexual characteristics, decreased muscle mass, and increased fat mass. Furthermore, hypogonadism in men is a risk factor for osteoporosis, metabolic syndrome, type II diabetes and cardiovascular disease.

Various testosterone replacement therapies are commercially available for the treatment of male hypogonadism. Pharmaceutical preparations include both testosterone and testosterone derivatives in the form of intramuscular injections, implants, oral tablets of alkylated T (e.g., methyltestosterone), topical gels, topical patches, or an intranasal gel. Over time, however, the current methods of treating testosterone deficiency suffer from poor compliance and thus unsatisfactory treatment of men with low T. For example, in a recently published study, patient adherence to topical T replacement therapy at 6 months was only 34.7% and by 12 months, only 15.4% of patients continued on topical T therapy (Medication Adherence and Treatment Patterns for Hypogonadal Patients Treated with Topical Testosterone Therapy: A Retrospective Medical Claims Analysis. Michael Jay Schoenfeld, Emily Shortridge, Zhanglin Cui and David Muram, Journal of Sexual Medicine March 2013).

Various testosterone replacement therapies are commercially available for the treatment of male hypogonadism. Pharmaceutical preparations include both testosterone and testosterone derivatives in the form of intramuscular injections, implants, oral tablets of alkylated T (e.g., methyltestosterone), topical gels, topical patches, or an intranasal gel.

Despite the advances that have been made in this field, there remains a need for new therapeutic products useful to treatment of testosterone deficiency. One such agent is testosterone undecanoate, which has the following chemical structure:

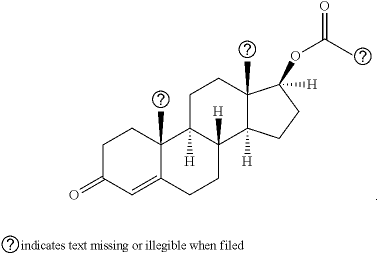

A formulation of testosterone undecanoate has been reported in the FDA approved drug label JATENZO®. The formulation also includes oleic acid, polyoxyl 40 hydrogenated castor oil (Cremophor RH 40), borage seed oil, peppermint oil, and butylated hydroxytoluene.

There is a significant, unmet need for methods for treating of testosterone deficiency. The present disclosure fulfills these and other needs, as evident in reference to the following disclosure.

Provided is a method of treating conditions associated with a deficiency or absence of endogenous testosterone in a subject in need thereof comprising the steps of:
administering to the subject a defined dose of an oral pharmaceutical composition comprising testosterone undecanoate solubilized in a carrier comprising at least one lipophilic surfactant and at least one hydrophilic surfactant;
collecting a blood sample from the subject;
measuring the serum testosterone concentration in the subject; and
increasing the dose of testosterone undecanoate when the measured serum testosterone concentration in the subject is less than about 425 ng/dl,
decreasing the dose of testosterone undecanoate when the measured serum testosterone concentration in the subject is greater than about 970 ng/dl, or
maintaining the dose of testosterone undecanoate when the measured serum testosterone concentration in the subject is between about 425 ng/dl and about 970 ng/dL.

Also provided is a method of treating chronic testosterone deficiency in a subject in need thereof comprising the steps of:
administering daily to the subject a defined dose of an oral pharmaceutical composition comprising testosterone undecanoate solubilized in a carrier comprising at least one lipophilic surfactant and at least one hydrophilic surfactant;
measuring the circulating testosterone concentration in the subject from which blood is collected; and
increasing the dose of testosterone undecanoate when the measured serum testosterone C₆ (i.e., scrum T concentration in a blood sample drawn 6 hours post an oral TU dose) in the subject is less than about 425 ng/dL, decreasing each dose of testosterone undecanoate administered when the serum testosterone C₆ in the subject is greater than about 970 ng/dL, and maintaining each dose of testosterone undecanoate administered when the measured serum testosterone C₆ in the subject is between about 425 ng/dl and about 970 ng/dL.

Also provided is a method of treating chronic testosterone deficiency in a subject in need thereof comprising the steps of:
administering daily to the subject a defined dose of an oral pharmaceutical composition comprising testosterone undecanoate solubilized in a carrier comprising at least one lipophilic surfactant and at least one hydrophilic surfactant;
collecting the subject's blood sample;
measuring the serum testosterone concentration in the subject;
making a titration decision based on the measured serum testosterone concentration; and
increasing the dose of testosterone undecanoate administered when the measured scrum testosterone concentration in the subject is less than about 425 ng/dL, decreasing each dose of testosterone undecanoate administered when the measured serum testosterone concentration in the subject is greater than about 970 ng/dL, and maintaining each dose of testosterone undecanoate administered when the measured serum testosterone concentration in the subject is between about 425 ng/dl and about 970 ng/dL.

Also provided is a method of treating chronic testosterone deficiency in a subject in need thereof comprising the steps of:
administering to the subject a first dose of an oral pharmaceutical composition comprising a 237 mg dose of testosterone undecanoate (TU) solubilized in a carrier comprising at least one lipophilic surfactant and at least one hydrophilic surfactant;
after a first time period, measuring the subject's testosterone concentration; and
increasing the dose of testosterone undecanoate administered when the measured scrum testosterone concentration is less than about 425 ng/dL, decreasing the dose of testosterone undecanoate administered when the measured serum testosterone concentration is greater than about 970 ng/dL, and maintaining the dose of testosterone undecanoate administered when the measured serum testosterone concentration in the subject is between about 425 ng/dl and about 970 ng/dL.

Also provided is a method of treating chronic testosterone deficiency in a subject in need thereof comprising the steps of:
administering twice daily (BID) to the subject an oral pharmaceutical composition comprising a 237 mg dose of testosterone undecanoate (TU) solubilized in a carrier comprising at least one lipophilic surfactant and at least one hydrophilic surfactant;
collecting the subject's blood sample six hours after administration of the morning dose;
measuring the serum testosterone concentration in the subject 6 hours after the morning dose; and
increasing the dose of testosterone undecanoate administered when the measured serum testosterone concentration in the subject is less than about 425 ng/dL, decreasing each dose of testosterone undecanoate administered when the measured scrum testosterone concentration in the subject is greater than about 970 ng/dL, and maintaining each dose of testosterone undecanoate administered when the measured scrum testosterone concentration in the subject is between about 425 ng/dl and about 970 ng/dL.

Also provided is a method of measuring a subject's testosterone concentration comprising:
collecting the subject's blood sample and obtaining the serum from the blood sample;
measuring the subject's scrum testosterone concentration; and
estimating the subject's blood testosterone concentration by measuring the subject's serum testosterone concentration and dividing that concentration by a conversion factor.

Disclosed herein is a method of treating conditions associated with a deficiency or absence of endogenous testosterone in a subject in need thereof comprising the steps of:
i. administering to the subject a defined dose of an oral pharmaceutical composition comprising testosterone undecanoate solubilized in a carrier comprising at least one lipophilic surfactant and at least one hydrophilic surfactant, wherein the defined dose is 237 mg administered twice daily;
ii. collecting a blood sample from the subject;
iii. measuring the serum testosterone concentration in the subject; and
iv. increasing the dose of testosterone undecanoate to 316 mg, administered twice daily, when the measured serum testosterone concentration in the subject is less than about 425 ng/dl,
v. decreasing the dose of testosterone undecanoate to 198 mg, administered twice daily, when the measured serum testosterone concentration in the subject is greater than about 970 ng/dL, or
vi. maintaining the dose of testosterone undecanoate when the measured serum testosterone concentration in the subject is between about 425 ng/dl and about 970 ng/dL.

Disclosed herein is a method of treating conditions associated with a deficiency or absence of endogenous testosterone in a subject in need thereof comprising the steps of:
i. administering to the subject a defined dose of an oral pharmaceutical composition comprising testosterone undecanoate solubilized in a carrier comprising at least one lipophilic surfactant and at least one hydrophilic surfactant, wherein the defined dose is 316 mg administered twice daily;
ii. collecting a blood sample from the subject;
iii. measuring the serum testosterone concentration in the subject; and
iv. increasing the dose of testosterone undecanoate to 396 mg, administered twice daily, when the measured serum testosterone concentration in the subject is less than about 425 ng/dl,
v. decreasing the dose of testosterone undecanoate to 237 mg, administered twice daily, when the measured serum testosterone concentration in the subject is greater than about 970 ng/dL, or
vi. maintaining the dose of testosterone undecanoate when the measured serum testosterone concentration in the subject is between about 425 ng/dl and about 970 ng/dL.

Disclosed herein is a method of treating conditions associated with a deficiency or absence of endogenous testosterone in a subject in need thereof comprising the steps of:
i. administering to the subject a defined dose of an oral pharmaceutical composition comprising testosterone undecanoate solubilized in a carrier comprising at least one lipophilic surfactant and at least one hydrophilic surfactant, wherein the defined dose is 198 mg administered twice daily;
ii. collecting a blood sample from the subject;
iii. measuring the serum testosterone concentration in the subject; and
iv. increasing the dose of testosterone undecanoate to 237 mg, administered twice daily, when the measured scrum testosterone concentration in the subject is less than about 425 ng/dl,
v. decreasing the dose of testosterone undecanoate to 158 mg, administered twice daily, when the measured serum testosterone concentration in the subject is greater than about 970 ng/dL, or
vi. maintaining the dose of testosterone undecanoate when the measured scrum testosterone concentration in the subject is between about 425 ng/dl and about 970 ng/dL.

Disclosed herein is a method of treating conditions associated with a deficiency or absence of endogenous testosterone in a subject in need thereof comprising the steps of:
i. administering to the subject a defined dose of an oral pharmaceutical composition comprising testosterone undecanoate solubilized in a carrier comprising at least one lipophilic surfactant and at least one hydrophilic surfactant, wherein the defined dose is 396 mg administered twice daily;
ii. collecting a blood sample from the subject;
iii. measuring the scrum testosterone concentration in the subject; and
iv. decreasing the dose of testosterone undecanoate to 316 mg, administered twice daily, when the measured serum testosterone concentration in the subject is greater than about 970 ng/dL, or
v. maintaining the dose of testosterone undecanoate when the measured scrum testosterone concentration in the subject is between about 425 ng/dL and about 970 ng/dL.

Disclosed herein is a method of treating conditions associated with a deficiency or absence of endogenous testosterone in a subject in need thereof comprising the steps of:
i. administering to the subject a defined dose of an oral pharmaceutical composition comprising testosterone undecanoate solubilized in a carrier comprising at least one lipophilic surfactant and at least one hydrophilic surfactant, wherein the defined dose is 158 mg administered twice daily;
ii. collecting a blood sample from the subject;
iii. measuring the scrum testosterone concentration in the subject; and
iv. increasing the dose of testosterone undecanoate to 198 mg, administered twice daily, when the measured serum testosterone concentration in the subject is less than about 425 ng/dL, and
v. treatment is discontinued when the subject's measured scrum testosterone concentration in the subject is greater than about 970 ng/dL.

In an embodiment, said scrum testosterone concentration is measured four to eight hours after administering the oral pharmaceutical composition.

In an embodiment, said oral pharmaceutical composition comprises about 19.8 percent by weight of solubilized testosterone undecanoate, about 51.6 percent by weight of oleic acid, and about 16.1 percent by weight of polyoxyethylene (40) hydrogenated castor oil.

In an embodiment, said oral pharmaceutical composition further comprises about 10 percent by weight of borage seed oil and about 2.5 percent by weight of peppermint oil.

In an embodiment, said oral pharmaceutical composition comprises about 15 percent by weight of solubilized testosterone undecanoate; about 16 percent by weight of polyoxyethylene (40) hydrogenated castor oil; about 63 percent by weight of glyceryl monolinoleate; and about 6 percent by weight of polyethylene glycol 8000.

In an embodiment, said testosterone concentrations are measured in serum from a blood sample collected in a plain tube drawn 5-7 hours after the morning dose and at least 7 days after starting treatment and following dose adjustment.

In an embodiment, said subject is diagnosed with hypogonadal conditions associated with structural or genetic etiologies.

In an embodiment, said subject is diagnosed with primary hypogonadism.

In an embodiment, said subject is diagnosed with testicular failure due to cryptorchidism, bilateral torsion, orchitis, vanishing testis syndrome, orchiectomy, Klinefelter syndrome, chemotherapy, or toxic damage from alcohol or heavy metals.

In an embodiment, said subject is diagnosed with hypogonadotropic hypogonadism.

In an embodiment, said subject is diagnosed with gonadotropin or lutcinizing hormone-releasing hormone (LHRH) deficiency or pituitary-hypothalamic injury from tumors, trauma, or radiation.

In an embodiment, said subject is not diagnosed with age-related hypogonadism.

In an embodiment, wherein prior to administering the defined dose of an oral pharmaceutical composition, further comprising the step of confirming the diagnosis of hypogonadism.

In an embodiment, said diagnosis of hypogonadism is confirmed if the serum testosterone concentrations measured on at least two separate days are below the normal range.

### BRIEF DESCRIPTION OF THE DRAWINGS

FIG. 1 shows a schematic of the difference in T concentrations measured in scrum and NaF-EDTA Plasma.

FIG. 2 shows the relationship between multiple effects on the T concentration and an overall conversion factor that can be used to translate the T concentration in one setting to an equivalent, actionable T concentration in a second setting.

### DETAILED DESCRIPTION

### Abbreviations and Definitions

To facilitate understanding, a number of terms and abbreviations as used herein are defined below as follows:

When introducing elements of the present invention or the particular embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

The term “about,” as used herein, is intended to qualify the numerical values that it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

The term “plasma,” as used herein, is intended to mean the liquid component of blood that holds the blood cells in whole blood in suspension; this makes plasma the extracellular matrix of blood cells. It makes up about 55% of the body's total blood volume. It is mostly water (up to 95% by volume), and contains dissolved proteins (6-8%) (i.e.—serum albumins, globulins, and fibrinogen), glucose, clotting factors, electrolytes (Na⁺, Ca²⁺, Mg²⁺, HCO₃⁻, Cl⁻, etc.), hormones, carbon dioxide (plasma being the main medium for excretory product transportation) and oxygen. This is in contrast to blood serum which is blood plasma without clotting factors. Further, plasma is derived from blood that is collected differently than when scrum is collected, by allowing the blood to clot prior to centrifugation when collecting scrum versus immediate centrifugation when collecting plasma.

The phrase “conditions associated with a deficiency or absence of endogenous testosterone” or “chronic testosterone deficiency,” as used herein, is intended to refer to:
Primary hypogonadism (congenital or acquired): testicular failure due to cryptorchidism, bilateral torsion, orchitis, vanishing testis syndrome, orchiectomy, Klinefelter syndrome, chemotherapy, or toxic damage from alcohol or heavy metals. These men usually have low serum testosterone concentrations and gonadotropins (follicle-stimulating hormone [FSH], luteinizing hormone [LH]) above the normal range; and
Hypogonadotropic hypogonadism (congenital or acquired): gonadotropin or luteinizing hormone-releasing hormone (LHRH) deficiency or pituitary-hypothalamic injury from tumors, trauma, or radiation. These men have low testosterone serum concentrations but have gonadotropins in the normal or low range.

The term “normal range” or “eugonadal range” as used herein, is intended to mean an average steady state plasma levels (concentrations) of testosterone of about 300-1100 ng/dL.

The term “lipophilic surfactant,” as used herein is intended to mean a surfactant having a hydrophilic-lipophilic balance (HLB) less than 10, such as less than 5, for example, 1 to 2. Certain lipophilic surfactants suitable in oral compositions described herein include fatty acids (C₆-C₂₄, such as C₁₀-C₂₄, for example C₁₄-C₂₄), for example, octanoic acid, decanoic acid, undecanoic acid, lauric acid, myristic acid, palmitic acid, palmitoleic, stearic acid, oleic acid, linoleic acid, alpha- and gamma- linolenic acid, arachidonic acid or combinations thereof. In some embodiments, the lipophilic surfactant is oleic acid. In some embodiments, the lipophilic surfactant is glyceryl monolinolcate.

Other lipophilic surfactants include:
Mono- and/or di-glycerides of fatty acids, such as glyceryl distearate, Imwitor 988 (glyceryl mono-/di-caprylate), Imwitor 742 (glyceryl mono-di-caprylate/caprate), Imwitor 308 (glyceryl mono-caprylate), Imwitor 191 (glyceryl mono-stearate), Softigen 701 (glyceryl mono-/di-ricinoleate), Capmul MCM (glyceryl caprylate/caprate), Capmul MCM(L) (liquid form of Capmul MCM), Capmul GMO (glyceryl mono-oleate), Capmul GDL (glyceryl dilaurate), Maisine (glyceryl mono-linoleate), Peceol (glyceryl mono-oleate), Myverol 18-92 (distilled monoglycerides from sunflower oil) and Myverol 18-06 (distilled monoglycerides from hydrogenated soybean oil), Precirol ATO 5 (glyceryl palmitostearate) and Gelucire 39/01 (semi-synthetic glycerides, i.e., C12-18 mono-, di-and tri-glycerides);
Acetic, succinic, lactic, citric and/or tartaric esters of mono- and/or di-glycerides of fatty acids, for example, Myvacet 9-45 (distilled acctylated monoglycerides), Miglyol 829 (caprylic/capric diglyceryl succinate), Myverol SMG (mono/di-succinylated monoglycerides), Imwitor 370 (glyceryl stearate citrate), Imwitor 375 (glyceryl monostearate/citrate/lactate) and Crodatem T22 (diacetyl tartaric esters of monoglycerides);
Propylene glycol mono- and/or di-esters of fatty acids, for example, Lauroglycol (propylene glycol monolaurate), Mirpyl (propylene glycol monomyristate), Captex 200 (propylene glycol dicaprylate/dicaprate), Miglyol 840 (propylene glycol dicaprylate/dicaprate) and Neobee M-20 (propylene glycol dicaprylate/dicaprate);
Polyglycerol esters of fatty acids such as Plurol oleique (polyglyceryl olcate), Caprol ET (polyglyceryl mixed fatty acids) and Drewpol 10.10.10 (polyglyceryl oleate);
Castor oil cthoxylates of low ethoxylate content (HLB<10) such as Etocas 5 (5 moles of ethylene oxide reacted with 1 mole of castor oil) and Sandoxylate 5 (5 moles of ethylene oxide reacted with 1 mole of castor oil;
Acid and ester ethoxylates formed by reacting ethylene oxide with fatty acids or glycerol esters of fatty acids (HLB<10) such as Crodet 04 (polyoxyethylene (4) lauric acid), Cithrol 2MS (polyoxyethylene (2) stearic acid), Marlosol 183 (polyoxyethylene (3) stearic acid) and Marlowet G12DO (glyceryl 12 EO dioleate). Sorbitan esters of fatty acids, for example, Span 20 (sorbitan monolaurate), Crill 1 (sorbitan monolaurate) and Crill 4 (sorbitan mono-oleate);
Transesterification products of natural or hydrogenated vegetable oil triglyceride and a polyalkylene polyol (HLB<10), e.g. Labrafil M1944CS (polyoxyethylated apricot kernel oil), Labrafil M2125CS (polyoxyethylated corn oil) and Gelucire 37/06 (polyoxyethylated hydrogenated coconut);
Alcohol ethyoxylates (HLB<10), e.g. Volpo N3 (polyoxyethylated (3) oleyl ether), Brij 93 (polyoxyethylated (2) oleyl ether), Marlowet LA4 (polyoxyethylated (4) lauryl ether); and
Pluronics, for example, Polyoxyethylene-polyoxypropylene co-polymers and block co-polymers (HLB<10) e.g. Synperonic PE L42 (HLB=8) and Synperonic PE L61 (HLB=3)

The term “hydrophilic surfactant,” as used herein is intended to mean a surfactant having an HLB of greater than 10, such as 10 to 45, for example, 10-15. In some embodiments, the hydrophilic surfactant is a polyoxyethylene sorbitan fatty acid ester, hydrogenated castor oil ethoxylate, PEG mono- and di-ester of palmitic and stearic acid, fatty acid ethoxylate, or combinations thereof. In some embodiments, the hydrophilic surfactant is selected from polyoxyethylene sorbitan fatty acid esters, hydrogenated castor oil ethoxylates, polyethylene glycol mono- and di- glycerol esters of caprylic, capric, palmitic and stearic acids, fatty acid ethoxylates, polyethylene glycol esters of alpha-tocopherol and its esters and combinations thereof. In some embodiments, the hydrophilic surfactant is a hydrogenated castor oil ethoxylate. In some embodiments, the hydrophilic surfactant is Cremophor RH 40 (polyoxyethyleneglycerol trihydroxystcarate).

Provided is a method of treating conditions associated with a deficiency or absence of endogenous testosterone in a subject in need thereof comprising the steps of:
administering to the subject a defined dose of an oral pharmaceutical composition comprising testosterone undecanoate solubilized in a carrier comprising at least one lipophilic surfactant and at least one hydrophilic surfactant;
collecting a blood sample from the subject;
measuring the serum testosterone concentration in the subject; and
increasing the dose of testosterone undecanoate when the measured serum testosterone concentration in the subject is less than about 425 ng/dl,
decreasing the dose of testosterone undecanoate when the measured serum testosterone concentration in the subject is greater than about 970 ng/dl, or
maintaining the dose of testosterone undecanoate when the measured serum testosterone concentration in the subject is between about 425 ng/dL and about 970 ng/dL.

Also provided is a method of treating chronic testosterone deficiency or its symptoms comprising the steps of:
administering daily to a subject in need thereof an oral pharmaceutical composition comprising 237 mg of testosterone undecanoate solubilized in a carrier comprising oleic acid, and polyoxyethyelene (40) hydrogenated castor oil, and optionally, borage seed oil and/or peppermint oil, twice a day, for a period of at least fourteen days;
collecting the subject's blood sample;
measuring the serum testosterone concentration in the subject five to seven hours following the daily administration of the oral pharmaceutical composition;
increasing the dose of testosterone equivalents administered daily to the subject by 50 mg when the serum testosterone concentration in the subject is less than 425 ng/dl, and decreasing the dose of testosterone equivalents administered daily to the subject by 25 mg when the scrum testosterone concentration in the subject is greater than 970 ng/dL; and
repeating until the serum testosterone concentration in the subject is between 425 and 970 ng/dL.

Also provided is a method of treating chronic testosterone deficiency in a subject in need thereof comprising the steps of:
administering daily to the subject a defined dose of an oral pharmaceutical composition comprising testosterone undecanoate solubilized in a carrier comprising at least one lipophilic surfactant and at least one hydrophilic surfactant;
measuring the circulating testosterone concentration in the subject from which blood is collected; and
increasing the dose of testosterone undecanoate administered when the measured serum testosterone C₆ (i.e., serum T concentration in a blood sample drawn 6 hours post an oral TU dose) in the subject is less than about 425 ng/dL, decreasing each dose of testosterone undecanoate administered when the serum testosterone C₆ in the subject is greater than about 970 ng/dL, and maintaining each dose of testosterone undecanoate administered when the measured serum testosterone C₆ in the subject is between about 425 ng/dl and about 970 ng/dL.

Also provided is a method of treating chronic testosterone deficiency in a subject in need thereof comprising the steps of:
administering daily to the subject a defined dose of an oral pharmaceutical composition comprising testosterone undecanoate solubilized in a carrier comprising at least one lipophilic surfactant and at least one hydrophilic surfactant;
collecting the subject's blood sample;
measuring the serum testosterone concentration in the subject;
making a titration decision based on the measured serum testosterone concentration; and
increasing the dose of testosterone undecanoate administered when the measured scrum testosterone concentration in the subject is less than about 425 ng/dL, decreasing each dose of testosterone undecanoate administered when the measured serum testosterone concentration in the subject is greater than about 970 ng/dL, and maintaining each dose of testosterone undecanoate administered when the measured serum testosterone concentration in the subject is between about 425 ng/dl and about 970 ng/dL.

Also provided is a method of treating chronic testosterone deficiency in a subject in need thereof comprising the steps of:
administering to the subject a first dose of an oral pharmaceutical composition comprising a 237 mg dose of testosterone undecanoate (TU) solubilized in a carrier comprising at least one lipophilic surfactant and at least one hydrophilic surfactant;
after a first time period, measuring the subject's testosterone concentration; and
increasing the dose of testosterone undecanoate administered when the measured serum testosterone concentration is less than about 425 ng/dL, decreasing the dose of testosterone undecanoate administered when the measured serum testosterone concentration is greater than about 970 ng/dL, and maintaining the dose of testosterone undecanoate administered when the measured serum testosterone concentration in the subject is between about 425 ng/dl and about 970 ng/dL.

Also provided is a method of treating chronic testosterone deficiency in a subject in need thereof comprising the steps of:
administering twice daily (BID) to the subject an oral pharmaceutical composition comprising a 237 mg dose of testosterone undecanoate (TU) solubilized in a carrier comprising at least one lipophilic surfactant and at least one hydrophilic surfactant;
collecting the subject's blood sample six hours after administration of the morning dose;
measuring the serum testosterone concentration in the subject 6 hours after the morning dose; and
increasing the dose of testosterone undecanoate administered when the measured serum testosterone concentration in the subject is less than about 425 ng/dL, decreasing each dose of testosterone undecanoate administered when the measured scrum testosterone concentration in the subject is greater than about 970 ng/dl, and maintaining each dose of testosterone undecanoate administered when the measured scrum testosterone concentration in the subject is between about 425 ng/dl and about 970 ng/dL.

In some embodiments, the steps are repeated until the serum testosterone concentration in the subject is between about 425 and about 970 ng/dL. In some embodiments, the steps are repeated until the serum testosterone concentration from blood collected 6 hours post-dose in the subject is between about 425 and about 970 ng/dL.

In some embodiments, the subject is diagnosed with hypogonadal conditions associated with structural or genetic etiologies. In some embodiments, the subject is diagnosed with primary hypogonadism. In some embodiments, the subject is diagnosed with testicular failure due to cryptorchidism, bilateral torsion, orchitis, vanishing testis syndrome, orchiectomy, Klinefelter syndrome, chemotherapy, or toxic damage from alcohol or heavy metals. In some embodiments, the subject is diagnosed with hypogonadotropic hypogonadism. In some embodiments, the subject is diagnosed with gonadotropin or luteinizing hormone-releasing hormone (LHRH) deficiency or pituitary-hypothalamic injury from tumors, trauma, or radiation. In some embodiments, the subject is not diagnosed with age-related hypogonadism.

In some embodiments, prior to administering the defined dose of an oral pharmaceutical composition, the method further comprises the step of confirming the diagnosis of hypogonadism. In some embodiments, the diagnosis of hypogonadism is confirmed if the scrum testosterone concentrations measured on at least two separate days are below the normal range.

In some embodiments, the oral pharmaceutical composition is administered twice daily (BID). In some embodiments, the oral pharmaceutical composition is administered three times daily (TID). In some embodiments, the oral pharmaceutical composition is administered once daily (QD).

In some embodiments, the oral pharmaceutical composition comprises testosterone undecanoate and is administered twice daily (BID). In some embodiments, the oral pharmaceutical composition comprises testosterone undecanoate and is administered three times daily (TID).

In some embodiments, the oral pharmaceutical composition is administered in close proximity to a meal (e.g., immediately prior or after a meal, or 15 minutes prior to after a meal or 30 minutes prior to or after a meal) wherein the meal contains at least about 15 g of fat.

In some embodiments, the meal contains at least about 30 g of fat.

In some embodiments, the meal contains at least about 45 g of fat.

In some embodiments, blood is drawn 4-8 hours (i.e., C₄₋₈) after administration of the dose. In some embodiments, blood is drawn 5-7 hours after administration of the dose. In some embodiments, blood is drawn 6 hours after administration of v dose.

In some embodiments, blood is drawn at least 7 days after starting treatment. In some embodiments, blood is drawn at least 7 days following dose adjustment.

In some embodiments, the subject's testosterone concentration is measured after a first time period. In some embodiments, the first period is chosen from 1, 2, 3, 4, 5, 6, 7, and 8 hours. In some embodiments, the first period is 4-8 hours. In some embodiments, the first period is 5-7 hours. In some embodiments, the first period is 6 hours.

In some embodiments, the plasma testosterone concentration is measured five to seven hours following the daily administration of the oral pharmaceutical composition. In some embodiments, the testosterone concentration is measured in serum from a blood sample collected in a plain tube drawn 5-7 hours after the morning dose. In some embodiments, the testosterone concentration is measured in serum from a blood sample collected in a plain tube drawn 5-7 hours after at least 7 days after starting treatment. In some embodiments, the testosterone concentration is measured in serum from a blood sample collected in a plain tube drawn 5-7 hours following dose adjustment.

In some embodiments, the serum testosterone C_{avg} is estimated on the basis of a single blood sample (i.e., C₆ serum T concentration). In some embodiments, the serum testosterone C_{avg} is estimated on the basis of a single blood sample collected 4 to 8 hours after administering the oral pharmaceutical composition. In some embodiments, the serum testosterone C_{avg} is estimated on the basis of a single blood sample collected 5 to 7 hours after administering the oral pharmaceutical composition. In some embodiments, the serum testosterone concentration at 6 hours after administration of the morning dose is used to approximate C_{avg}. In some embodiments, the scrum testosterone C_{avg} is estimated on the basis of a single blood sample collected 6 hours after administering the oral pharmaceutical composition.

In some embodiments, the serum testosterone C_{avg} determined based on the measurement of testosterone via an immunometric assay, or a liquid chromatography tandem mass spectrometry (LC-MS/MS) assay.

In some embodiments, the steady-state serum testosterone C_{avg} is estimated based on the measurement of testosterone in a single blood sample collected about 4 to 8 hours after oral testosterone dose after at least seven days of daily treatment with the oral pharmaceutical composition. In some embodiments, the steady-state serum testosterone C_{avg} is estimated based on the measurement of testosterone in a single blood sample collected about 5 to 7 hours after oral testosterone dose after at least seven days of daily treatment with the oral pharmaceutical composition. In some embodiments, the steady-state serum testosterone C_{avg} is estimated based on the measurement of T in a single blood sample collected about 6 hours after oral testosterone dose after at least seven days of daily treatment with the oral pharmaceutical composition.

In some embodiments, the serum testosterone C_{avg} is determined after at least 10 to 14 days of daily treatment with the oral pharmaceutical composition. In some embodiments, the scrum testosterone C_{avg} is determined after at least 30 days of daily treatment with the oral pharmaceutical composition.

In some embodiments, the circulating testosterone concentration is measured in serum. In some embodiments, the serum testosterone concentration is measured four to eight hours after administering the oral pharmaceutical composition.

Also provided is a method of measuring a subject's testosterone concentration comprising: collecting the subject's blood sample and obtaining the serum from the blood sample; measuring the subject's serum testosterone concentration; and estimating the subject's blood testosterone concentration by measuring the subject's scrum testosterone concentration and dividing that concentration by a conversion factor.

In some embodiments, measuring the subject's testosterone concentration comprises collecting the subject's blood sample and obtaining the serum from the blood sample. In some embodiments, measuring the subject's testosterone concentration is comprises measuring the subject's serum testosterone concentration. In some embodiments, measuring the subject's testosterone concentration comprises estimating the subject's blood testosterone concentration by measuring the subject's serum testosterone concentration and dividing that concentration by a conversion factor.

In some embodiments, the conversion factor is between about 1.0 and about 1.4. In some embodiments, the conversion factor is between about 1.032 and about 1.396.

In some embodiments, the conversion factor is 1.214. In some embodiments, if the sample is a serum sample, then dividing the T concentration measured in serum by 1.214 would yield the expected T concentration in NaF-EDTA plasma.

In some embodiments, the incidence of concordant titration decisions is approximately 99%.

In some embodiments, concordant titration decisions reflect an on-diagonal concordance of 75% and 63% and an effective off-diagonal titration decision of 24% and 36%.

In some embodiments, the defined dose is about 237 mg of testosterone undecanoate, administered twice daily.

In some embodiments, the 237 mg dose of testosterone undecanoate is increased to 316 mg, administered twice daily, when the measured scrum testosterone concentration in the subject is less than about 425 ng/dL and the dose of testosterone undecanoate is decreased to 198 mg, administered twice daily, when the measured serum testosterone concentration in the subject is greater than about 970 ng/dL.

In some embodiments, the defined dose is about 316 mg of testosterone undecanoate, administered twice daily. In some embodiments, the defined dose is about 316 mg of testosterone undecanoate, administered twice daily and the dose of testosterone undecanoate is increased to 396 mg, administered twice daily, when the measured scrum testosterone concentration in the subject is less than about 425 ng/dL; and the dose of testosterone undecanoate is decreased to 237 mg, administered twice daily, when the measured serum testosterone concentration in the subject is greater than about 970 ng/dL.

In some embodiments, the defined dose is about 198 mg of testosterone undecanoate, administered twice daily. In some embodiments, the defined dose is about 198 mg of testosterone undecanoate, administered twice daily and the dose of testosterone undecanoate is increased to 237 mg, administered twice daily, when the measured serum testosterone concentration in the subject is less than about 425 ng/dL; and the dose of testosterone undecanoate is decreased to 158 mg, administered twice daily, when the measured scrum testosterone concentration in the subject is greater than about 970 ng/dL.

In some embodiments, the defined dose is about 396 mg of testosterone undecanoate, administered twice daily. In some embodiments, the defined dose is about 396 mg of testosterone undecanoate, administered twice daily and the dose of testosterone undecanoate is decreased to 316 mg, administered twice daily, when the measured serum testosterone concentration in the subject is greater than about 970 ng/dL.

In some embodiments, the defined dose is about 158 mg of testosterone undecanoate, administered twice daily. In some embodiments, the defined dose is about 158 mg of testosterone undecanoate, administered twice daily and the dose of testosterone undecanoate is increased to 198 mg, administered twice daily, when the measured serum testosterone concentration in the subject is less than about 425 ng/dL; and treatment is discontinued when the subject's measured serum testosterone concentration in the subject is greater than about 970 ng/dL.

In some embodiments, the dose adjustment scheme is as shown in Table 1.

TABLE 1

| Testosterone Concentration in | | |
|---|---|---|
| Serum From Plain (Red-Top) | Current Dose | New Dose |
| Tube Drawn 6 hours After Dose | (mg, BID) | (mg, BID) |
| <425 ng/dL | 158 | 198 |
| | 198 | 237 |
| | 237 | 316 |
| | 316 | 398 |
| 425-970 ng/dL | No Dose Change | |
| >970 ng/dL | 396 | 316 |
| | 316 | 237 |
| | 237 | 198 |
| | 198 | 158 |
| | 158 | Discontinue Treatment |

In some embodiments, the initial dose of testosterone undecanoate in the oral pharmaceutical composition is equivalent to about 150 mg of testosterone. In some embodiments, the oral pharmaceutical composition comprises testosterone undecanoate. In some embodiments, the oral pharmaceutical composition administered comprises about 237 mg of testosterone undecanoate that equates to 150 mg of testosterone.

In some embodiments, the initial dose of testosterone undecanoate in the oral pharmaceutical composition is equivalent to about 200 mg of testosterone per dose. In some embodiments, the oral pharmaceutical composition comprises testosterone undecanoate. In some embodiments, the oral pharmaceutical composition administered comprises about 316 mg of testosterone undecanoate that equates to 200 mg testosterone per dose.

In some embodiments, the initial dose of testosterone undecanoate in the oral pharmaceutical composition is equivalent to about 250 mg of testosterone per dose. In some embodiments, the oral pharmaceutical composition comprises testosterone undecanoate. In some embodiments, the oral pharmaceutical composition administered comprises about 396 mg of testosterone undecanoate that equates to 250 mg testosterone per dose.

In some embodiments, the initial dose of testosterone undecanoate in the oral pharmaceutical composition is equivalent to about 125 mg of testosterone per dose. In some embodiments, the oral pharmaceutical composition comprises testosterone undecanoate. In some embodiments, the oral pharmaceutical composition administered comprises about 198 mg of testosterone undecanoate that equates to 125 mg testosterone per dose.

In some embodiments, the initial dose of testosterone undecanoate in the oral pharmaceutical composition is equivalent to about 100 mg of testosterone per dose. In some embodiments, the oral pharmaceutical composition comprises testosterone undecanoate. In some embodiments, the oral pharmaceutical composition administered comprises about 158 mg of testosterone undecanoate that equates to 100 mg testosterone per dose.

In some embodiments, the dose of testosterone undecanoate in the administered oral pharmaceutical composition is increased by the equivalent of about 25 to about 75 mg of testosterone when the serum testosterone C₆ in the subject is less than about 425 ng/dL, and decreased by the equivalent of about 10 to about 75 mg of testosterone when the scrum testosterone C₆ in the subject is greater than about 970 ng/dL.

In some embodiments, the dose of testosterone undecanoate in the administered oral pharmaceutical composition is increased by the equivalent of about 40 to about 60 mg of testosterone when the serum testosterone C₆ in the subject is less than about 425 ng/dL.

In some embodiments. the dose of testosterone undecanoate in the administered oral pharmaceutical composition is increased by the equivalent of about 50 mg of testosterone when the serum testosterone C₆ in the subject is less than about 425 ng/dL.

In some embodiments, the dose of testosterone undecanoate in the administered oral pharmaceutical composition is decreased by the equivalent of about 10 to about 60 mg of testosterone when the serum testosterone Ce in the subject is greater than about 970 ng/dL.

In some embodiments, the dose of testosterone undecanoate in the administered oral pharmaceutical composition is decreased by the equivalent of about 25 to about 50 mg of testosterone when the serum testosterone Co in the subject is greater than about 970 ng/dL.

In some embodiments, the dose of testosterone undecanoate in the administered oral pharmaceutical composition is decreased by the equivalent of about 25 mg of testosterone when the serum testosterone C₆ in the subject is greater than about 970 ng/dL.

In some embodiments, the dose of oral pharmaceutical composition is titrated after 21 days of daily treatment. In some embodiments, the dose of oral pharmaceutical composition is titrated after 56 days of daily treatment. In some embodiments, the dose of oral pharmaceutical composition is titrated after 105 days of daily treatment.

In some embodiments, the dose of oral pharmaceutical composition is titrated after at least 30 days of daily treatment. In some embodiments, the dose of oral pharmaceutical composition is titrated after 35 days of daily treatment. In some embodiments, the dose of oral pharmaceutical composition is titrated after at least 60 days of daily treatment. In some embodiments, the dose of oral pharmaceutical composition is titrated after 70 days of daily treatment.

In some embodiments, the oral pharmaceutical composition comprises testosterone undecanoate solubilized in a carrier comprising at least one lipophilic surfactant and at least one hydrophilic surfactant in a total lipophilic surfactant to total hydrophilic surfactant ratio (w/w) falling in the range of about 6:1 to 3.5:1, which composition, upon once- or twice-daily oral administration, provides an average scrum testosterone concentration at steady state falling in the range of about 425 to about 970 ng/dL.

In some embodiments, the composition comprises 15-30% (w/w) of testosterone undecanoate.

In some embodiments, the composition comprises 15-20% (w/w) of testosterone undecanoate.

In some embodiments, the composition comprises 18-22% (w/w) of testosterone undecanoate. In some embodiments, the oral pharmaceutical composition comprises about 18 to 22 percent by weight of a solubilized testosterone undecanoate.

In some embodiments, the composition comprises 25-30% (w/w) of testosterone undecanoate.

In some embodiments, the oral pharmaceutical composition comprises about 10-20 percent by weight of solubilized testosterone undecanoate, about 5-20 percent by weight of hydrophilic surfactant, about 50-70 percent by weight of lipophilic surfactant; and about 10-15 percent by weight of digestible oil, wherein the oral pharmaceutical composition is free of ethanol.

In some embodiments, oral pharmaceutical compositions comprise testosterone undecanoate solubilized in a carrier in a lipophilic surfactant and a hydrophilic surfactant. The compositions are designed to be self-emulsifying drug delivery systems (SEDDS) and iterations thereof such as self-microemulsified drug delivery systems (SMEDDS) and self-nanoemulsified drug delivery systems (SNEDDS) so that a testosterone undecanoate -containing emulsion, microemulsion, nanoemulsion (or dispersion) is formed upon mixing with intestinal fluids in the gastrointestinal tract.

In some embodiments, the oral pharmaceutical composition comprises 15 to 17 percent by weight of the at least one hydrophilic surfactant.

In some embodiments, the oral pharmaceutical composition comprises 50 to 55 percent by weight of the at least one lipophilic surfactant.

In some embodiments, the oral pharmaceutical composition further includes digestible oil. A digestible oil is defined herein as an oil that is capable of undergoing de-esterification or hydrolysis in the presence of pancreatic lipase in vivo under normal physiological conditions. Specifically, digestible oils may be complete glycerol triesters of medium chain (C₇-C₁₃) or long chain (C₁₄-C₂₂) fatty acids with low molecular weight (up to C₆) mono-, di- or polyhydric alcohols. Some examples of digestible oils for use the oral pharmaceutical composition include: vegetable oils (e.g., soybean oil, safflower seed oil, corn oil, olive oil, castor oil, cottonseed oil, arachis oil, sunflower seed oil, coconut oil, palm oil, rapeseed oil, black currant oil, evening primrose oil, grape seed oil, wheat germ oil, sesame oil, avocado oil, almond, borage, peppermint and apricot kernel oils) and animal oils (e.g., fish liver oil, shark oil and mink oil). In some embodiments, the digestible oil is a vegetable oil. In some embodiments, the vegetable oil is soybean oil, safflower seed oil, corn oil, olive oil, castor oil, cottonseed oil, arachis oil, sunflower seed oil, coconut oil, palm oil, rapeseed oil, evening primrose oil, grape seed oil, wheat germ oil, sesame oil, avocado oil, almond oil, borage oil, peppermint oil, apricot kernel oil, or combinations thereof. Particular digestible oils are those with high gamma-linolenic acid (GLA) content such as, black currant oil, primrose oil and borage oil, as well as any other digestible oil that can be enriched in GLA acid through enzymatic processes.

In some embodiments, methods and compositions for modulating (i.e., sustaining) the rate of available serum testosterone by incorporating component(s) that may biochemically modulate (1) testosterone ester absorption. (2) testosterone ester metabolism to testosterone. and/or (3) metabolism of testosterone to dihydrotestosterone (DHT). For example, the inclusion of medium to long chain fatty acid esters can enhance testosterone ester absorption. In this way, more testosterone ester may stave off hydrolysis in the gut and enter the blood stream. In other words, the fatty acid ester may competitively inhibit esterases that would otherwise metabolize the testosterone ester. Examples of other esters or combinations thereof include botanical extracts or benign esters used as food additives (e.g., propylparaben, octylacetate and ethylacetate).

Other components that can modulate testosterone ester absorption include “natural” and synthetic inhibitors of 5α-reductase, which is an enzyme present in enterocytes and other tissues that catalyzes the conversion of T to DHT. Complete or partial inhibition of this conversion may both increase and sustain increased plasma levels of T after oral dosing with testosterone ester while concomitantly reducing plasma DHT levels. Borage oil, which contains a significant amount of the 5a-reductase inhibitor, gamma-linolenic acid (GLA), is an example of a “natural” modulator of testosterone ester metabolism. Other than within borage oil, of course. GLA could be added directly as a separate component. Furthermore, any digestible oil as listed above can be enzymatically enriched in GLA. Many natural inhibitors of 5a-reductase are known in the art (e.g., epigallocatechin gallate, a catechin derived primarily from green tea and saw palmetto extract from berries of the Serenoa repens species, phytosterols and lycopene), all of which may be suitable in the compositions described herein. Non-limiting examples of synthetic 5α-reductase inhibitors include compounds such as finasteride, dutasteride and the like.

In some embodiments. the oral pharmaceutical composition further includes one or more additional therapeutic agents. In some embodiments, the agent is a second testosterone ester. a synthetic progestin, an inhibitor of type-I and/or type II 5α-reductase, an inhibitor of CYP3A4, finasteride, dutasteride, thiazide diuretics, and calcium channel blockers, or combinations thereof. In some embodiments, the agent is borage oil. In some embodiments, the agent is peppermint oil and related substances such as menthol and menthol esters. In some embodiments, the agent is a second testosterone ester.

In some embodiments, the thiazide diuretic is selected from chlorothiazide, chlorthalidone, indapamide, hydrochlorothiazide, methyclothiazide, and metolazone.

In some embodiments, the calcium channel blocker is selected from Amlodipine, Diltiazem, Felodipine, Isradipine, Nicardipine, Nifedipine, Nisoldipine, and Verapamil.

Optional cosolvents suitable with the oral pharmaceutical composition are, for example, water, short chain mono-, di-, and polyhydric alcohols, such as ethanol, benzyl alcohol, glycerol, propylene glycol, propylene carbonate, polyethylene glycol (PEG) with an average molecular weight of about 200 to about 10,000, diethylene glycol monoethyl ether (e.g., Transcutol HP), and combinations thereof. In particular, such cosolvents, especially monohydric alcohols. are excluded altogether. Thus, in some embodiments, the oral pharmaceutical compositions are free of monohydric alcohols. In some embodiments, the monohydric alcohols are C₂-C₁₈ aliphatic or aromatic alcohols. In some embodiments, the compositions are free of ethyl or benzyl alcohols.

In some embodiments, the compositions contain between 0% and 10% (w/w) of polyethylene glycol with an average molecular weight of about 8,000 (PEG-8000). In some embodiments, the compositions contain between 5% and 10% (w/w) of PEG-8000.

In some embodiments. the oral pharmaceutical composition is a liquid, semi-solid or solid dosage form. In some embodiments, the oral pharmaceutical compositions are liquid or semi-solid at ambient temperatures. Furthermore, these pharmaceutical compositions can be transformed into solid dosage forms through adsorption onto solid carrier particles, such as silicon dioxide, calcium silicate or magnesium aluminometasilicate to obtain free-flowing powders that can be either filled into hard capsules or compressed into tablets. Hence, the term “solubilized” herein, should be interpreted to describe an active pharmaceutical ingredient (API), which is dissolved in a liquid solution or which is uniformly dispersed in a solid carrier. In addition, sachet type dosage forms can be formed and used. In some embodiments, the oral pharmaceutical composition is filled into a hard or soft gelatin capsule.

In some embodiments. the oral pharmaceutical composition comprises: about 15-20 percent by weight of solubilized testosterone undecanoate, about 5-20 percent by weight of hydrophilic surfactant, about 50-70 percent by weight of lipophilic surfactant; and about 1-10 percent by weight of polyethylene glycol 8000. In some embodiments, the oral pharmaceutical composition comprises about 15 percent by weight of testosterone undecanoate, about 63 percent by weight of glyceryl mono-linoleate, about 16 percent by weight of polyoxyethylene (40) hydrogenated castor oil, and about 6 percent by weight of polyethylene glycol having a molecular weight of about 8000 g/mol (PEG 8000).

The compositions details of Table 2 (mg/capsule and wt. percentage) are based on an approximate fill weight of 800 mg fill weight per ‘00’ hard gelatin capsule. However, at testosterone undecanoate amounts less than about 100 mg/capsule, the formulations may be proportionally adjusted for smaller total fill weights that would permit use of smaller hard gelatin capsules (e.g., size ‘0’ or smaller size if needed).

As well, it should be apparent to one of ordinary skill in the art that many, if not all, of the surfactants within a category (e.g., lipophilic, hydrophilic, etc.) may be exchanged with another surfactant from the same category. Thus, while many of the formulations comprise oleic acid, one of ordinary skill in the art should recognize other lipophilic surfactants (e.g., those listed above) may be suitable as well. Similarly, while Table 2 lists formulations comprising Cremophor RH40 (HLB=13), one of ordinary skill in the art should recognize other hydrophilic surfactants (e.g., those listed above) may be suitable. Borage oil, peppermint oil, BHT, and ascorbyl palmitate may be substituted for chemically similar substances or eliminated.

TABLE 2

| | Composition % w/w (mg/ “00” capsule)¹ | | | | | | | Fill |
|---|---|---|---|---|---|---|---|---|
| | | Oleic | Cremophor | Borage | Peppermint | | Ascorbyl | Wt. |
| F. | TU | Acid | RH40 | Oil | Oil | BHT | Palmitate | (mg)² |
| 1 | 20 | 51.5 | 16 | 10 | 2.5 | 0.06 | — | 800 |
| | (158) | (413) | (128.5) | (80) | (20) | (0.5) | | |
| 2 | 15 | 54.5 | 18 | 10 | 2.5 | 0.02 | 0.8 | 806.6 |
| | (120) | (436) | (144) | (80) | (20) | (0.2) | (6.4) | |
| 3 | 17 | 52.5 | 18 | 10 | 2.5 | 0.02 | 0.8 | 806.6 |
| | (136) | (420) | (144) | (80) | (20) | (0.2) | (6.4) | |
| 4 | 19 | 50.5 | 18 | 10 | 2.5 | 0.02 | 0.8 | 806.6 |
| | (152) | (404) | (144) | (80) | (20) | (0.2) | (6.4) | |
| 5 | 21 | 50 | 16.5 | 10 | 2.5 | 0.02 | 0.8 | 806.6 |
| | (168) | (400) | (132) | (80) | (20) | (0.2) | (6.4) | |
| 6 | 23 | 50 | 14.5 | 10 | 2.5 | 0.02 | 0.8 | 806.6 |
| | (184) | (400) | (116) | (80) | (20) | (0.2) | (6.4) | |
| 7 | 25 | 50 | 12.5 | 10 | 2.5 | 0.02 | 0.8 | 806.6 |
| | (200) | (400) | (100) | (80) | (20) | (0.2) | (6.4) | |
| 8 | 16 | 53.5 | 18 | 10 | 2.5 | 0.02 | 0.8 | 806.6 |
| | (128) | (428) | (144) | (80) | (20) | (0.2) | (6.4) | |
| 9 | 18 | 51.5 | 18 | 10 | 2.5 | 0.02 | 0.8 | 806.6 |
| | (144) | (413) | (144) | (80) | (20) | (0.2) | (6.4) | |
| 10 | 22 | 50 | 15.5 | 10 | 2.5 | 0.02 | 0.8 | 806.6 |
| | (176) | (400) | (124 | (80) | (20) | (0.2) | (6.4) | |
| 11 | 24 | 50 | 13.5 | 10 | 2.5 | 0.02 | 0.8 | 806.6 |
| | (192) | (400) | (108) | (80) | (20) | (0.2) | (6.4) | |
| 12 | 15 | 55.5 | 17 | 10 | 2.5 | 0.02 | 0.8 | 806.6 |
| | (120) | (444) | (136) | (80) | (20) | (0.2) | (6.4) | |
| 13 | 17 | 53.5 | 17 | 10 | 2.5 | 0.02 | 0.8 | 806.6 |
| | (136) | (428) | (136) | (80) | (20) | (0.2) | (6.4) | |
| 14 | 19 | 51.5 | 17 | 10 | 2.5 | 0.02 | 0.8 | 806.6 |
| | (152) | (412) | (136) | (80) | (20) | (0.2) | (6.4) | |
| 15 | 15 | 56.5 | 16 | 10 | 2.5 | 0.02 | 0.8 | 806.6 |
| | (120) | (452) | (128) | (80) | (20) | (0.2) | (6.4) | |
| 16 | 17 | 54.5 | 16 | 10 | 2.5 | 0.02 | 0.8 | 806.6 |
| | (136) | (436) | (128) | (80) | (20) | (0.2) | (6.4) | |
| 17 | 19 | 52.5 | 16 | 10 | 2.5 | 0.02 | 0.8 | 806.6 |
| | (152) | (420) | (128) | (80) | (20) | (0.2) | (6.4) | |
| 18 | 21 | 50.5 | 16 | 10 | 2.5 | 0.02 | 0.8 | 806.6 |
| | (168) | (404) | (128) | (80) | (20) | (0.2) | (6.4) | |
| 19 | 20 | 50.5 | 17 | 10 | 2.5 | 0.02 | 0.8 | 806.6 |
| | (160) | (404) | (136) | (80) | (20) | (0.2) | (6.4) | |
| 20 | 20 | 51.5 | 16 | 10 | 2.5 | 0.02 | 0.8 | 806.6 |
| | (160) | (412) | (128) | (80) | (20) | (0.2) | (6.4) | |
| 21 | 15 | 57.5 | 15 | 10 | 2.5 | 0.02 | 0.8 | 806.6 |
| | (120) | (460) | (120) | (80) | (20) | (0.2) | (6.4) | |
| 22 | 16 | 56.5 | 15 | 10 | 2.5 | 0.02 | 0.8 | 806.6 |
| | (128) | (452) | (120) | (80) | (20) | (0.2) | (6.4) | |
| 23 | 17 | 55.5 | 15 | 10 | 2.5 | 0.02 | 0.8 | 806.6 |
| | (136) | (444) | (120) | (80) | (20) | (0.2) | (6.4) | |
| 24 | 18 | (54.5 | 15 | 10 | 2.5 | 0.02 | 0.8 | 806.6 |
| | (144) | (436) | (120) | (80) | (20) | (0.2) | (6.4) | |
| 25 | 19 | 53.5 | 15 | 10 | 2.5 | 0.02 | 0.8 | 806.6 |
| | (152) | (428) | (120) | (80) | (20) | (0.2) | (6.4) | |
| 26 | 20 | 51.5 | 16 | 9.4 | 3.1 | 0.06 | — | 800 |
| | (158) | (413) | (128.5) | (75) | (25) | (0.5) | | |
| 27 | 20 | 51.5 | 16 | 10.6 | 1.9 | 0.06 | — | 800 |
| | (158) | (413) | (128.5) | (85) | (15) | (0.5) | — | |
| 28 | 20 | 51.5 | 16 | 11.2 | 1.2 | 0.02 | 0.8 | 806.1 |
| | (158) | (413) | (128.5) | (90) | (10) | (0.2) | (6.4) | |
| 29 | 20 | 51.5 | 16 | 11.8 | 0.6 | 0.02 | 0.8 | 806.1 |
| | (158) | (413) | (128.5 | (95) | (5) | (0.2) | (6.4) | |
| 30 | 25 | 50 | 12.5 | 10.6 | 1.9 | 0.06 | — | 800.5 |
| | (200) | (400) | (100) | (85) | (15) | (0.5) | | |

¹Milligram weights rounded to nearest whole number; 800 (±10%)
²±8 mg

In some embodiments, the oral pharmaceutical composition comprises about 19.8 percent by weight of solubilized testosterone undecanoate, about 51.6 percent by weight of oleic acid, about 16.1 percent by weight of polyoxyethylene (40) hydrogenated castor oil, about 10 percent by weight of borage seed oil, about 2.5 percent by weight of peppermint oil, and about 0.03 percent by weight of butylated hydroxytoluene (BHT).

Particular formulations of TU filled into size “00” capsules are:

Formulation A

| Ingredients | mg/capsule | %, w/w |
|---|---|---|
| Testosterone Undecanoate | 158.3 | 19.8 |
| Oleic Acid | 413.1 | 51.6 |
| Cremophor RH 40 | 128.4 | 16.1 |
| Borage Seed Oil | 80.0 | 10 |
| Peppermint Oil | 20.0 | 2.5 |
| BHT | 0.2 | 0.03 |
| Total | 800 | 100 |

Formulation B

| Ingredients | mg/capsule | %, w/w |
|---|---|---|
| Testosterone Undecanoate | 158.3 | 19.8 |
| Oleic Acid | 412.5 | 51.6 |
| Cremophor RH 40 | 128.4 | 16.0 |
| Peppermint Oil | 20.0 | 2.5 |
| Borage Seed Oil + 0.03% BHT | 80.0 | 10 |
| Ascorbyl Palmitate | 0.8 | 0.1 |
| Total | 800 | 100 |

Formulation C

| Ingredients | mg/capsule | %, w/w |
|---|---|---|
| Testosterone Undecanoate | 120 | 15 |
| Cremophor RH 40 | 128 | 16 |
| Maisine 35-1 | 504 | 63 |
| Polyethylene Glycol 8000 | 48 | 6 |
| TOTAL | 800 | 100 |

In some embodiments, the oral pharmaceutical composition comprises about 19.8 percent by weight of solubilized testosterone undecanoate, about 51.6 percent by weight of oleic acid, and about 16.1 percent by weight of polyoxyethylene (40) hydrogenated castor oil. In some embodiments, the oral pharmaceutical composition further comprises about 10 percent by weight of borage seed oil and about 2.5 percent by weight of peppermint oil. In some embodiments, the oral pharmaceutical composition comprises about 15 percent by weight of solubilized testosterone undecanoate; about 16 percent by weight of polyoxyethylene (40) hydrogenated castor oil; about 63 percent by weight of glyceryl monolinoleate; and about 6 percent by weight

In some embodiments, the oral pharmaceutical composition exhibits a percent (%) in vitro dissolution profile in 5% Triton X-100 solution in phosphate buffer, pH 6.8, indicating release from the composition of substantially all of the solubilized testosterone undecanoate within about 2 hours.

In some embodiments, the oral pharmaceutical composition exhibits a percent (%) in vitro dissolution profile in 5% Triton X-100 solution in phosphate buffer, pH 6.8, indicating release from the composition of substantially all of the solubilized testosterone undecanoate within about 1 hour.

### EXAMPLES

### Baseline T Concentrations

Baseline concentrations of T were determined prior to the start of the study and immediately prior to the start of each treatment cycle (i.e., after each 7 to 14-day washout period). The washout periods were sufficiently long to assure that T concentrations from the previous dosing cycle were no longer detectable.

### Example

### Influence of Blood Collection Tubes

Testosterone undecanoate is metabolized into testosterone. Its degradation in whole blood into testosterone has been studied in conditions typically used in clinical trials. It was observed that TU degrades extensively to testosterone in human blood under conditions typical of harvesting serum, causing overestimation of testosterone concentration.

Historically, most testosterone monitoring for diagnostic purposes and for testosterone replacement therapy (TRT) dose titration has been based on the testosterone concentration in blood concentrations in tubes without additives. However, many of the FDA approved immunoassays can accurately measure testosterone in serum as well as EDTA-plasma, or heparin-plasma. For subjects receiving oral testosterone undecanoate (TU), it has been proposed that monitoring of blood concentrations should be done with tubes that contain a nonspecific esterase inhibitor, sodium fluoride (NaF). Collecting the blood samples in tubes containing NaF may influence the blood testosterone concentration. Namely, use of NaF when oral TU is administered will enable a more accurate assay of true circulating T concentration.

As previously noted, despite the inaccuracy of measuring testosterone in scrum in men treated with oral testosterone esters, specifically with oral TU, the desirability of the serum matrix and blood handling requirements for immunoassays for testosterone still make serum a desirable medium for measurement of testosterone. Collecting samples in NaF-EDTA tubes which are processed on ice is not convenient in the clinical setting and the plasma is not a validated matrix for immunoassays; however, measuring the T in this plasma allows for accurate measurements of T which are necessary in clinical trials, such as those required by regulatory agencies. It is therefore necessary to develop a method of deriving a way to correlate a scrum testosterone levels (plain tube collected and allowed to clot for 30 minutes before centrifugation to collect serum) with a T level measured in NaF-EDTA plasma level (NaF-EDTA tube collected and allowed to sit for 30 minutes on ice before centrifugation to collect plasma).

Each study participant has blood drawn and the study drug (comprising 396 mg TU) is dispensed, followed by administration of the first study drug dose immediately prior to a breakfast meal containing 30 g fat. Prior to discharge from the clinical facility, the subjects will be instructed to take 396mg TU by mouth immediately prior to their dinner meal on Day 1, then twice daily on Days 2-6 immediately prior to their breakfast and dinner meals, and on Day 7 in the morning, immediately prior to their breakfast meal.

Subjects will return to the Clinic on Day 7 in the late afternoon (before dinner) with their study medication. Upon arrival at the clinic, their study drug will be counted to determine compliance during Day 1 through Day 7 post-AM dose. They will then be administered the study drug 396 mg TU by mouth immediately prior to a dinner meal (containing 30 g fat) which will be provided. Subjects will be instructed to consume the dinner in approximately 20 minutes. Subjects will remain in the unit overnight.

On the 8^{th} day, 5 hrs after their AM meal, blood is collected in plain, EDTA and NaF-EDTA tubes. The plain tubes sit at room temperature (RT). The EDTA and NaF-EDTA tubes are placed on ice. The blood is spun (centrifuged) at various intervals after it is drawn (0, 15, 30, 60, 90 and 120 minutes, except plain which do not have a 0- and 15-minute sample).

Samples will be collected into multiple Plain, EDTA and NaF-EDTA tubes. The Plain tube will be incubated at RT to allow blood clotting, as recommended by Clinical and Laboratory Standards Institute (CLSI 2010). EDTA and NaF-EDTA tubes will be held on ice. After a defined period (between zero and 120 minutes), the tube(s) will be centrifuged and the serum/plasma transferred into freezer vial/tube(s) and frozen. The study site was given specific instructions on sample handling. The handling instructions were specific to the sample collection lube type and are presented in the following paragraphs.

Plain Collection Tube—Incubated at room temperature (scrum): Collected blood tubes are labelled with the incubation time points listed described above and are incubated at room temperature for a minimum of 30 minutes. Centrifugation earlier than 30 minutes is not appropriate since the blood will not be fully clotted. Each sample will be centrifuged at the specified time point for 20 minutes at >1000 g. For each blood collection tube, serum will be separated promptly after centrifugation and transferred into appropriately-labelled polypropylene tubes. The serum samples will then be stored at −20° C. (±5° C.) prior to analysis.

NaF-EDTA Tubes—Incubated in ice bath (plasma): Collected blood tubes are labelled with the incubation time points listed described above and incubated in an ice bath for the specified incubation time points and then centrifuged for 20 minutes at >1000 g. For each blood collection tube, plasma will be separated promptly after centrifugation and transferred into appropriately-labelled polypropylene tubes. The plasma samples will then be stored at −20° C. (±5° C.) prior to analysis.

The concentration of total T and TU will be determined in the serum/plasma samples of all subjects using validated LC/MS/MS methods at Syneos Health Laboratories located in Quebec City, Quebec, Canada. The analytical procedures will be conducted in compliance with the laboratory's Standard Operating Procedures.

In order to calculate the ex vivo increase in T concentrations due to TU to T conversion, it is necessary to know the initial T concentration (e.g., T concentration in circulation). When calculating the ex vivo TU to T conversion in experiments where TU is spiked into blood, the unspiked blood's T concentration serves as the true baseline. When TU to T conversion is calculated in experiments where oral TU is administered, then the closest approximation to the “true” baseline value is the T concentration in a blood sample which is processed immediately after phlebotomy (e.g., centrifuged at time=0). Since samples in Plain tubes need 30 minutes to clot, it is not possible to get a “true” baseline value for scrum. Therefore, as a surrogate for a serum “true” baseline, the Day 8 EDTA “true” baseline (time =0) will be used.

One of the goals of this study was to determine whether the T concentration in blood samples collected into Plain tubes (yielding serum) can be used for making dose titration decisions. In previous studies, dose titration decisions were based on T concentrations in NaF-EDTA plasma. Dose titration decisions were based on a 24 hr C_{avg}, but concordance analysis demonstrated that a sample collected about six hours post-morning dose can approximate the patient's C_{avg} and thus effectively guide dose titration.

When comparing the T concentration measured in NaF-EDTA tubes with that in Plain tubes, it is necessary to consider 2 factors. These 2 factors are the effect that NaF has on T levels and the effect of post-collection TU to T conversion has on T levels.

First, NaF negatively impacts the measurement of T concentrations (Wang 2008, Lachance 2015, Ceponis 2018) by LC-MS/MS (i.e., the true value of T is higher than when measured in the presence of NaF). The NaF effect was demonstrated not to be related to an impact on the assay itself (LC-MS/MS) but rather an effect on the partitioning of T into the plasma and the cellular components of blood when NaF is added to blood before it is centrifuged. In the presence of NaF, some T partitions into blood cells and this fraction is not available for measurement in plasma.

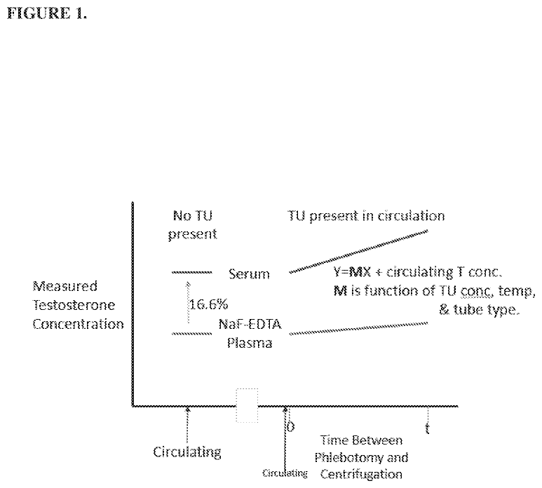

A second factor to consider when comparing T concentrations in serum and NaF-EDTA plasma measured in men taking oral TU, is the post-collection (i.e., ex vivo) conversion of TU to T by the action of non-specific esterases in blood. This post-collection conversion increases the T measured in the sample compared to the actual T concentration in the subject's circulation. Because the TU to T conversion is an enzymatic reaction mediated by esterases, its rate (extent) is determined by the TU concentration in the blood sample, the temperature of the sample (e.g., RT or on ice), the duration that the sample sits before centrifugation, and the presence of esterase inhibitors in the blood sample (e.g., whether drawing the blood into a tube which contains NaF). This is schematically presented in FIG. 1 which shows that measured T concentrations are different between scrum and NaF-EDTA plasma.

The TU concentration in the blood sample depends on the PK profile of the oral TU formulation (and thus is formulation specific) as well as how long after dosing the sample is collected.

The study explored the impact of the variables that determine the slope (M) in FIG. 1, particularly regarding the extent to which they depended on temperature and tube type. TU concentration was not controlled, but the blood samples will be drawn at a proposed dose-titration sample time, and so approximate the clinical setting for titration of oral TU. Based on the analysis, the difference between the T concentration between serum and EDTA-plasma was estimated for particular sample handling conditions. This difference allowed the determination of a correction factor for T concentration for any particular handling situation studied and the T concentration in NaF-EDTA plasma, EDTA plasma, and scrum collected in plain tubes.

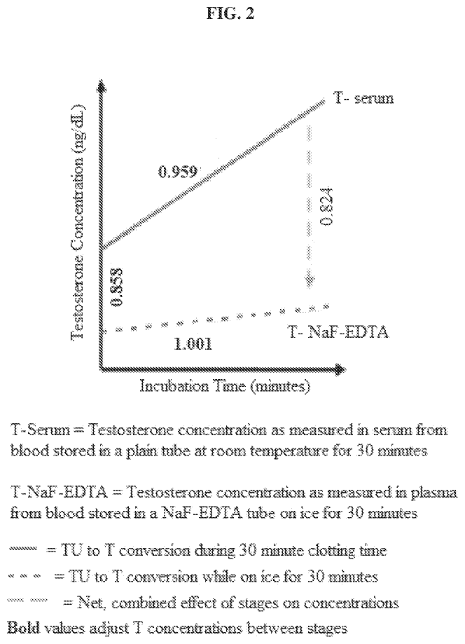

As shown in FIG. 2, the blue arrows indicate the steps of the conversion for a sample collected 6 hours post-dose in plain tube (allowed to clot at room temperature for 30 minutes) into the expected T concentration if the sample had been collected in a NaF-EDTA tube (and kept on ice for 30 minutes). First, the T concentration in the serum (from the plain tube) is reduced by the factor compensating for TU-to-T conversion at room temperature for 30 minutes (0.959, as indicated by the top arrow). Second, the T concentration is further reduced by the matrix effect associated with NaF-EDTA plasma compared to serum (0.858). Third, the T concentration is increased by the Tu-to-T conversion that occurs when a sample in a NaF-EDTA tube is help on ice for 30 minutes (1.001)

The combined effect (0.959×0.858×1.001=0.824) is the product of the three effects known to affect serum concentration. The expected value of the equivalent T concentration in plasma from a NaF-EDTA tube is 82.4% of the serum value when the incubation temperature, incubation time, and underlying TU concentration are as stated. The reciprocal value (1/0.858=1.214) can be used to convert a NaF-EDTA plasma T concentration into an equivalent scrum concentration.

The correction factor allows a T concentration measured in, for example, a plain tube held for 30 minutes at RT to be converted into a corresponding NaF-EDTA plasma concentration which was held for example for 30 minutes on ice. The correction factor is specific for given post-dose time or time-period, since it depends on the TU concentration expected in the blood sample.

Based on the experiment described, to convert a T concentration from a 6 hour (i.e., C₆) post-dose sample measured in NaF-EDTA plasma (isolated after blood tube sat on ice for 30 minutes) to the T concentration expected in serum (isolated after blood tube sat at room temperature for 30 minutes), the NaF-EDTA plasma T concentration is multiplied by 1.214. Also, if the sample had been a serum sample, then dividing the T concentration measured in serum by 1.214 would yield the expected T concentration in NaF-EDTA plasma. This ‘correction factor’ was accounted for in calculating efficacy in the study because the cugonadal range was one which was developed for T levels measured in NaF-EDTA plasma, namely 252-907 ng/dL, and not the ‘typical’ cugonadal range for serum, namely 300-1000 ng/dL. Thus, when converting the ‘typical’ cugonadal range for serum collected in plain tubes, the NaF-EDTA plasma range should have the correction factor applied to it, which leads to a ‘typical’ range of 306-1101 ng/dL. This ‘corrected’ value can then be used for dose titration decisions.

For the 6 hr sample, going from NaF-EDTA plasma to serum the conversion factor is 1.214 (95% confidence interval: 1.088, 1.340). Going from serum to NaF-EDTA plasma the correction factor is 0.824 (95% confidence interval: 0.743, 0.904).

The conversion factor only has to be about 1.214. In this case about means a difference of less that +15% or 1.032 to 1.396. This degree of difference is the variability allowed in assay validation such as described in the FDA's Bioanalytic Method Validation Guidance for Industry published in May 2018.

### Dose Titration

The goal of dose-titration in men treated with oral TU is to bring the testosterone C_{avg} into the cugonadal range. Because titration decisions based on testosterone C_{avg} are not practical in real world clinical setting, single sample T concentrations can be used as surrogates of C_{avg}. Concordance analysis estimates how well dose titration based on two different measures (e.g., C_{avg} v. C₆) work in guiding dose titration. On-diagonal titration indicates that the two measures result in the same dose-titration decision. However, to determine total concordance, effective off-diagonal titration also must be determined. Effective off-diagonal titration can be estimated for the following reasons. First, testosterone exposure is dose proportional, so it is possible to predict the change in C_{avg} with change in dose. Second, the titration boundaries (425 to 970 ng/dL) fall within the cugonadal boundaries (300 to 1100 ng/dL), and the cugonadal range is wide (3.5 fold) compared to the largest dose increment (33%) or decrement (25%). This means that the dose increments/decrements employed can allow movement within the eugonadal range (e.g. increasing the dose of someone with a C_{avg} of 400 ng/dL will raise the C_{avg} to a maximum of 532 ng/dL [400×1.33]). Similarly, when titration decisions based on C₆ are different from those based on C_{avg}, the outcome will often be a C_{avg} in the eugonadal range. For example, when a patient's C₆ is less than 425 ng/dl (indicating a dose increase is required), but whose C_{avg} is 600 ng/dL (indicating no titration), the impact of titrating based on C₆ is that the C_{avg} will increase but remain in the cugonadal range. The largest dose increase will result in a 33% increase in exposure which, in this case, will raise the C_{avg} to 798 ng/dL. Therefore, despite titration based on C₆, this patient's C_{avg} is not likely to rise above the upper boundary of the eugonadal range. Thus, the titration decision based on C₆ is effectively concordant with that based on C_{avg}, since both titration decisions will result in a patient with a C_{avg} in the eugonadal range. Selected cells in Table 3 indicate a patient's C_{avg} that would result in effective off-diagonal titration. Therefore, when comparing the effectiveness of dose-titration decisions based on C₆ and C_{avg}, both concordance (on-diagonal agreement between C₆ and C_{avg}) and effective off-diagonal agreement must be considered) [i.e., total concordance (percent of patients treated with oral TU who fall into given concordance boxes) is defined as the sum of on-diagonal and effective off-diagonal concordance (see area in Table 3 defined by heavy line). In the example presented, the C₆ value is a serum value derived from a measured NaF-EDTA plasma value and a conversion factor that enables the conversion of NaF-EDTA plasma data to be converted to an equivalent serum data.

TABLE 3

| | | C_{avg} calculated from NaF-EDTA plasma | | |
|---|---|---|---|---|
| Titration boundaries | | <350 ng/dL | 350 - 800 ng/dl. | >800 ng/dl |
| Serum | < 425 ng/dL | Concordant | Patients with C_{avg} ≤ 682 ng/dl | Discordant |
| C₆ | Increase dose | | remain in eugonadal range | |
| | (maximum 33%) | | after dose titration | |
| | 425 - 970 ng/dl | Patients with | Concordant | Discordant″ |
| | No dose change | Cary ≥ 252 ng/dl are in | | |
| | | eugonadal range | | |
| | > 970 ng/dl. | Discordant^{a} | All patients remain in | Concordant |
| | Decrease dose | | eugonadal range despite | |
| | (maximum 25%) | | decrease in dose | |

Abbreviations:
C₆ = concentration 6 hours after morning dose; C_{avg} = average observed concentration
over 24 hours
Note:
Concordance: when the titration decision based on C₆ or C_{avg} was the same. Off-Diagonal
Titration Decision: when the titration decision based on C₆ results in a dose that will generate a C_{avg}
in the cugonadal range.
^{a}Although some cases may have effective off-diagonal titrations, these cases are so rare as to not
impact the overall concordance.

An analysis of data derived from hypogonadal men treated with oral TU demonstrates that for certain clinic visits, the incidence of appropriate (i.e. concordant) titration decisions approximately 99% (Table 4). These reflect an on-diagonal concordance of 75% and 63% and an effective off-diagonal titration decision of 24% and 36% for Visit 2 and Visit 4b, respectively. Therefore, dose titration based on C₆ can effectively adjust a patient's oral TU dose such that his C_{avg} is in the eugonadal range.

TABLE 4
Visit 2 (Concordant + Effective Off-Diagonal Titration = 98.7%)

| | | Cag from NaF-EDTA Plasma | | | |
|---|---|---|---|---|---|
| | Titration | | | | |
| | boundaries | <350 ng/dL | 350 - 800 ng/dl. | >800 ng/dl | Total |
| C₆ | < 425 ng/dl | 59.4% | 13.9% | 0% | 73.3% |
| derived | Increase dose | | (ET = 13.9%) | | |
| | (maximum 33 %) | | | | |
| from | 425 - 970 ng/dL | 5.4% | 15.2% | 2% | 22.6% |
| NaF- | No dose change | (L'T = 4.2%) | | (ET =1.2 %) | |
| EDTA | > 970 ng/dl. | 0% | 4.2% | 0.6% | 4.8% |
| plasma | Decrease dose | | (ET = 4.2%) | | |
| | (maximum 25 %) | | | | |
| value | Total | 64.8% | 33.3% | 2.6% | 100% |

Visit 4b (Concordant + Effective Off-Diagonal Titration = 99.1% )

| | | C_{avg} from NaF-EDTA Plasma | | | |
|---|---|---|---|---|---|
| | Titration | | | | |
| | boundaries | <350 ng/dI. | 350 - 800 ng/dl | >800 ng/dl | Total |
| C₆ | <425 ng/dl | 37.5% | 21.8% | 0% | 59.3% |
| derived | Increase dose | | (ET = 21.8%) | | |
| | (maximum 33 %) | | | | |
| from | 425 - 970 ng/dl | 10% | 25% | 0.6% | 35.6% |
| NaF- | No dose change | (ET = 9.3%) | | (ET = 0.6%) | |
| EDTA | >970 ng/dL | 0% | 4.3% | 0.6% | 4.9% |
| plasma | Decrease dose | | (ET = 4.3%) | | |
| | (maximum 25%) | | | | |
| value | Total | 47.5% | 51.1% | 1.2% | 100% |

Abbreviations:
C₆ = concentration 6 hours after morning dose;
C_{avg} = average observed concentration over 24 hours;
ET = Effective Off-Diagonal Titration Decision

A single sample drawn 6 hours after the AM dose can effectively guide dose titration. The titration decision agreement (concordance plus effectiveness of off-diagonal titration decision) between C₆ and C_{avg} was high (about 99%) at the 2 titration visits.

### Other Embodiments

The detailed description set-forth above is provided to aid those skilled in the art in practicing the present invention. However, the invention described and claimed herein is not to be limited in scope by the specific embodiments herein disclosed because these embodiments are intended as illustration of several aspects of the invention. Any equivalent embodiments are intended to be within the scope of this invention. Indeed, various modifications of the invention in addition to those shown and described herein will become apparent to those skilled in the art from the foregoing description, which do not depart from the spirit or scope of the present inventive discovery. Such modifications are also intended to fall within the scope of the appended claims.

1-30. (canceled)

31. A method of treating one or more conditions associated with a deficiency or absence of endogenous testosterone in a subject, comprising:
administering a defined dose of testosterone undecanoate to the subject in an oral pharmaceutical composition twice daily, once in the morning and once in the evening, wherein the defined dose is 237 mg of testosterone undecanoate;
measuring serum testosterone concentration in the subject at steady state after at least seven days of daily administration of the defined dose and 6 hours after administration of a morning dose; and
either:
increasing the defined dose of testosterone undecanoate to 316 mg when the measured serum testosterone concentration is less than about 425 ng/dL, maintaining the defined dose of testosterone undecanoate when the measured serum testosterone concentration is between about 425 ng/dl and about 970 ng/dL; or
decreasing the defined dose of testosterone undecanoate to 198 mg when the measured serum testosterone concentration is greater than about 970 ng/dL;
wherein the oral pharmaceutical composition comprises the testosterone undecanoate solubilized in a carrier comprising at least one lipophilic surfactant and at least one hydrophilic surfactant.

32. The method of claim 31, wherein the measuring is performed from serum collected in a plain tube.

33. The method of claim 31, wherein the one or more conditions are selected from primary hypogonadism and hypogonadotropic hypogonadism.

34. The method of claim 33, wherein the one or more conditions is hypogonadotropic hypogonadism caused by gonadotropin or luteinizing hormone-releasing hormone (LHRH) deficiency or pituitary-hypothalamic injury from tumors, trauma, or radiation.

35. The method of claim 31, wherein the defined dose of testosterone undecanoate is increased to 316 mg and the method further comprises:
remeasuring the serum testosterone concentration in the subject at steady state after at least seven days of daily administration of the increased defined dose of 316 mg and 6 hours after administration of a morning dose; and
either:
increasing the defined dose of testosterone undecanoate to 396 mg when the serum testosterone concentration is less than about 425 ng/dL;
maintaining the defined dose of testosterone undecanoate when the measured serum testosterone concentration is between about 425 ng/dl and about 970 ng/dL; or
decreasing the defined dose of testosterone undecanoate to 237 mg when the serum testosterone concentration is greater than about 970 ng/dL.

36. The method of claim 31, wherein the defined dose of testosterone undecanoate is decreased to 198 mg and the method further comprises:
remeasuring the serum testosterone concentration in the subject at steady state after at least seven days of daily administration of the decreased defined dose of 198 mg and 6 hours after administration of a morning dose; and either:
increasing the defined dose of testosterone undecanoate to 237 mg when the serum testosterone concentration is less than about 425 ng/dL;
maintaining the defined dose of testosterone undecanoate when the measured serum testosterone concentration is between about 425 ng/dL and about 970 ng/dL; or
decreasing the defined dose of testosterone undecanoate to 158 mg when the serum testosterone concentration is greater than about 970 ng/dL.

37. The method of claim 31, wherein the ratio (w/w) in the composition of total lipophilic surfactant to total hydrophilic surfactant is in the range of about 6:1 to about 3.5:1.

38. The method of claim 31, wherein the lipophilic surfactant is a fatty acid.

39. The method of claim 31, wherein the hydrophilic surfactant is a hydrogenated castor oil ethoxylate.

40. The method of claim 31, wherein the oral pharmaceutical composition comprises a digestible oil.

41. The method of claim 31, wherein the lipophilic surfactant is oleic acid and the hydrophilic surfactant is polyoxyethylene (40) hydrogenated castor oil.

42. The method of claim 41, wherein the oral pharmaceutical composition further comprises borage seed oil and peppermint oil.

43. The method of claim 31, wherein the oral pharmaceutical composition comprises about 18-22% (w/w) testosterone undecanoate.

44. The method of claim 43, wherein the oral pharmaceutical composition comprises about 50-70% (w/w) lipophilic surfactant and about 5-20% (w/w) hydrophilic surfactant.

45. The method of claim 44, wherein the oral pharmaceutical composition comprises about 50-70% (w/w) oleic acid and about 5-20% (w/w) polyoxyethylene (40) hydrogenated castor oil.

46. The method of claim 45, wherein the oral pharmaceutical composition comprises about 50-70% (w/w) oleic acid, about 5-20% (w/w) polyoxyethylene (40) hydrogenated castor oil and about 10-15% (w/w) digestible oil.

47. The method of claim 46, wherein the oral pharmaceutical composition comprises about 50-70% (w/w) oleic acid, about 5-20% (w/w) polyoxyethylene (40) hydrogenated castor oil and about 10-15% (w/w) digestible oil comprising borage seed oil and peppermint oil.
